(19) United States
(12) Patent Application Publication
(10) Pub. No.: US 2025/0207042 A1
(43) Pub. Date: Jun. 26, 2025

(54) PROCESS FOR CONVERTING NAPHTHA TO LIGHT PARAFFINS WITH STAGED REACTORS

(71) Applicant: UOP LLC, Des Plaines, IL (US)

(72) Inventors: John J. Senetar, Naperville, IL (US); Scott Lyle Nauert, Chicago, IL (US)

(21) Appl. No.: 18/975,684

(22) Filed: Dec. 10, 2024

Related U.S. Application Data
(60) Provisional application No. 63/613,019, filed on Dec. 20, 2023.

(51) Int. Cl. C10G 47/00; C10G 55/06

(52) U.S. Cl. CPC C10G 47/00; C10G 55/06; C10G 2300/1037; C10G 2400/20

(57) ABSTRACT

A process for converting naphtha to ethane and propane is disclosed. The process comprises separating a naphtha stream into a plurality of naphtha streams. Each of the plurality of naphtha streams and a hydrogen stream are contacted with a catalyst in a dedicated one of a plurality of reactors to produce a plurality of contacted streams. One of the contacted streams can be separated into an ethane stream and a propane stream.

### FIELD

The field is the conversion of naphtha to light paraffins. The field may particularly relate to converting naphtha to ethane and propane.

### BACKGROUND

Light olefin production is vital to the production of sufficient plastics to meet worldwide demand. Dehydrogenation (PDH) is a process in which light paraffins such as ethane and propane can be dehydrogenated to make ethylene and propylene, respectively typically in the presence of a catalyst. Dehydrogenation can be achieved in either the presence of an oxidant such as oxygen or in the absence of an oxidant. Non-oxidative dehydrogenation is an endothermic reaction which requires external heat to drive the reaction to completion. Propane dehydrogenation (PDH) is a widely practiced example of non-oxidative dehydrogenation to produce propylene from propane. Ethane oxidative dehydrogenation is a newer oxidative process for converting ethane to ethylene which can be conducted at lower temperatures with lower carbon oxide emissions than non-oxidative and thermal cracking processes.

Fluid catalytic cracking (FCC) is another endothermic process that can be tuned to produce substantial propylene. However, not every FCC unit is tuned to make substantial propylene. Also, high propylene FCC units do not recover much ethylene; less than 1% of global ethylene supply comes from FCC.

The great bulk of the ethylene consumed in the production of plastics and petrochemicals such as polyethylene is produced by the thermal cracking of hydrocarbons. Steam is usually mixed with the feed stream to the cracking furnace to reduce the hydrocarbon partial pressure and enhance olefin yield and to reduce the formation and deposition of carbonaceous material in the cracking reactors. The process is therefore often referred to as steam cracking or pyrolysis.

Paraffins with a range of carbon numbers can be thermally cracked to produce olefins including ethane, propane, butanes, and naphtha. Ethane and naphtha feeds are typical due to higher light olefin yield than propane and butane feeds. Ethane feed is used in regions where light hydrocarbon gases are prevalent. In regions, where gas is not abundant, naphtha feed is employed for steam cracking. Naphtha steam cracking has long set the price in the ethylene industry due to higher production cost versus ethane steam cracking. The world does not currently produce enough ethane to supply the growing demand for ethylene. Therefore, regions lacking ethane supply such as Asia and Europe rely mainly on naphtha steam cracking to supply ethylene. Naphtha steam cracking yields only about 30%-35% ethylene with the balance including both relatively high-value by-products comprising propylene, butadiene, and butene-1 and relatively low value by-products comprising pyoil, pygas, and fuel gas. Additional pressures on naphtha steam cracking including minimum production requirements and environmental concerns have led to the withholding of government approvals in certain regions such as China. The ethylene industry needs a more efficient, economical and environmentally friendly route to light olefins from naphtha feeds.

### BRIEF SUMMARY

A process for converting naphtha to light olefins is disclosed. The process comprises separating a naphtha stream into a plurality of naphtha streams. Each of the plurality of naphtha streams are contacted with a catalyst in a dedicated one of a plurality of reactors to produce a plurality of contacted streams. One or more of the plurality of contacted streams can be separated into an ethane stream and a propane stream.

### BRIEF DESCRIPTION OF THE DRAWINGS

FIGURE is a schematic drawing of a process and apparatus of the present disclosure.

### DEFINITIONS

The term “communication” means that fluid flow is operatively permitted between enumerated components, which may be characterized as “fluid communication”.

The term “downstream communication” means that at least a portion of fluid flowing to the subject in downstream communication may operatively flow from the object with which it fluidly communicates.

The term “upstream communication” means that at least a portion of the fluid flowing from the subject in upstream communication may operatively flow to the object with which it fluidly communicates.

The term “direct communication” means that fluid flow from the upstream component enters the downstream component without passing through any other intervening vessel.

The term “indirect communication” means that fluid flow from the upstream component enters the downstream component after passing through an intervening vessel.

As used herein, the term “predominant” or “predominate” means greater than 50%, suitably greater than 75% and preferably greater than 90%.

The term “Cx” is to be understood to refer to molecules having the number of carbon atoms represented by the subscript “x”. Similarly, the term “Cx−” refers to molecules that contain less than or equal to x and preferably x and less carbon atoms. The term “Cx+” refers to molecules with more than or equal to x and preferably x and more carbon atoms.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column. Stripper columns may omit a reboiler at a bottom of the column and instead provide heating requirements and separation impetus from a fluidized inert media such as steam. Stripping columns typically feed a top tray and take the main product from the bottom.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure.

### DETAILED DESCRIPTION

C3-C8 hydrocarbon feed stock is primarily charged to a “Naphtha to Ethane and Propane” unit to convert naphtha in the presence of hydrogen into desirable ethane and propane along with less desirable methane. “Naphtha to ethane and propane” is a highly exothermic process. Without management of the exotherm, the temperature may become unacceptably high leading to inoperability of the process and poor yields. The negative impacts of high temperature include rapid catalyst deactivation, low selectivity to ethane and propane, high selectivity to undesirable byproducts such as methane and aromatics, a high rate of coke formation, and metallurgical concerns such as mechanical strength and coking tendency.

The current process provides interstage or interbed addition of naphtha feed. The process comprises separating a naphtha stream into a plurality of naphtha streams. Each of the plurality of naphtha streams is then passed to a dedicated one of a plurality of reactors. The naphtha stream and a hydrogen stream are contacted with a catalyst in the dedicated reactors to produce a plurality of contacted streams. One or more of the plurality of contacted streams can be separated into an ethane stream and a propane stream. The benefit of staging naphtha includes higher ethane to propane ratio, higher hydrogen conversion, and low aromatics selectivity.

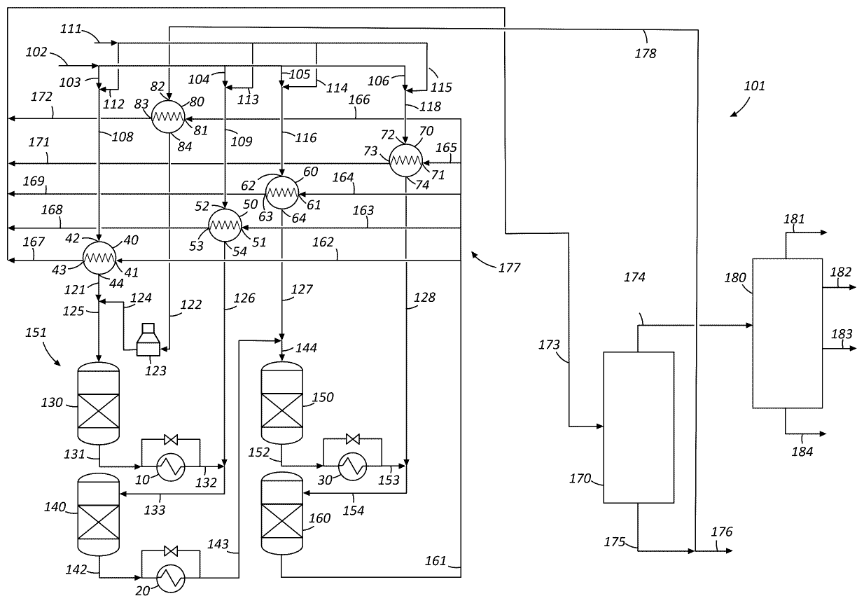

Turning to FIG. 1, an embodiment of a process and an apparatus for converting naphtha to ethane and propane 101 is disclosed. The process comprises a NEP reactor 151, a heat exchanger system 177, a splitter 170, and a separation unit 180. In an aspect, the NEP reactor 151 may comprise a multistage NEP reactor. In an exemplary embodiment shown in the FIGURE, the NEP reactor 151 may comprise four reactors, a first NEP reactor 130, a second NEP reactor 140, a third NEP reactor 150, and a fourth NEP reactor 160. A naphtha stream in line 102 may be combined with a hydrogen stream in line 111 to provide a charge stream which is passed to the NEP reactor 151. In accordance with the present disclosure, the naphtha stream in line 102 may be separated into a plurality of naphtha streams and passed to a dedicated NEP reactor of the multistage reactor 151. In an exemplary embodiment, the naphtha stream in line 102 may be separated into four naphtha streams a first naphtha stream in line 103, a second naphtha stream in line 104, a third naphtha stream in line 105, and a fourth naphtha stream in line 106. In another aspect, a hydrogen stream in line 111 may be separated into a plurality of hydrogen streams to provide a dedicated hydrogen stream for each of the naphtha stream and the NEP reactors. In accordance with another exemplary embodiment, the hydrogen stream in line 111 may be separated into four hydrogen streams, a first hydrogen stream in line 112, a second hydrogen stream in line 113, a third hydrogen stream in line 114, and a fourth hydrogen stream in line 115. The dedicated hydrogen streams may be combined with the dedicated naphtha streams to provide a dedicated charge stream for each of the plurality of reactors.

As shown in FIGURE, the first naphtha stream in line 103 may be combined with the first hydrogen stream in line 112 to provide a dedicated first charge stream in line 108. The first charge stream in line 108 is passed to the dedicated first reactor 130. Similarly, the second naphtha stream in line 104 may be combined with the second hydrogen stream in line 113 to provide a dedicated second charge stream in line 109, the third naphtha stream in line 105 may be combined with the third hydrogen stream in line 114 to provide a dedicated third charge stream in line 116, and the fourth naphtha stream in line 106 may be combined with the fourth hydrogen stream in line 115 to provide a dedicated fourth charge stream in line 118. In an aspect, the first charge stream in line 108, the second charge stream in line 109, the third charge stream in line 116, and the fourth charge stream in line 118 may be heated in the heat exchanger system 177 by heat exchange with a reactor effluent stream before charging to the dedicated reactors. In an embodiment, the heat exchanger system 177 may comprise a plurality of dedicated heat exchangers for heating each of the dedicated naphtha streams or the dedicated charge streams. In an exemplary embodiment, the heat exchanger system 177 may comprise four dedicated heat exchangers, a first heat exchanger 40, a second heat exchanger 50, a third heat exchanger 60, and a fourth heat exchanger 70.

The first charge stream in line 108 is passed to the first heat exchanger 40 to provide a first heated charge stream in line 121. The first heat exchanger 40 may comprise a hot inlet side 41 and a cold outlet side 43 for the hot stream, and a cold inlet side 42 and a hot outlet side 44 for the cold stream. A hot contacted stream is passed to the hot inlet side 41 and after heat exchange is removed from the cold outlet side 43 of the first heat exchanger 40. The first charge stream in line 108 is passed to the cold inlet side 42 and after heat exchange, the first heated charge stream is removed from the hot outlet side 44 of the first heat exchanger 40 in line 121. As described hereinafter in detail, the first heated charge stream in line 121 is combined with a recycle stream in line 124 to provide a first combined charge stream in line 125. The first combined charge stream in line 125 is passed to the first NEP reactor 130. The first naphtha stream and the first hydrogen stream are contacted with an NEP catalyst in the first NEP reactor 130 to provide a first contacted stream.

In accordance with the present disclosure, the naphtha stream may comprise C4 to C12 hydrocarbons preferably having a T10 between about −10° C. and about 60° C. and a T90 between about 70 and about 180° C. The naphtha feed stream may comprise normal paraffins, olefins, iso-paraffins, naphthenes, and aromatics. The naphtha stream may be heated to a reaction temperature of about 300° C. to about 600° C., suitably between about 325° C. and about 550° C., and preferably between about 350° C. and about 525° C. Overall weight space velocity, defined herein as hourly mass flow rate of feed in stream 102 divided by total catalyst in all reactors should be between about 0.3 to about 20 hr⁻¹, suitably between about 0.5 and about 10 hr⁻¹ and preferably between about 1 to about 4 hr⁻¹. A total pressure should be about 0.1 to about 3 MPa (abs), or from about 1.5 to about 2.5 MPa (abs), preferably greater than 1 MPa (abs). In an embodiment, the first NEP reactor 130 may be operated at a higher pressure than the other reactors. Under these conditions, C2-C4 yield is consistently in an excess of 80 wt %, while methane yield is less than about 16 wt %, suitably below about 14 wt % and typically below about 12 wt % and preferably no more than 10 wt %.

In accordance with the present disclosure, the first NEP reactor 130, the second NEP reactor 140, the third NEP reactor 150, and the fourth NEP reactor 160 may be operated at similar or different operating conditions.

The hydrogen-to-hydrocarbon molar ratio is important to selectively producing ethane and propane. The hydrogen-to-hydrocarbon ratio should be about 0.3 to about 15 and preferably about 0.5 to about 5. In a further embodiment, the hydrogen-to-hydrocarbon molar ratio may typically be no more than 5, suitably be no more than 3 and preferably be no more than 2. Low hydrogen-to-hydrocarbon ratio promotes desired reaction kinetics which are initiated with dehydrogenation. Hydrogen-to-hydrocarbon ratio may range from about 50% to about 500%, suitably no more than 300% and preferably no more than 200% of stoichiometric requirements to convert naphtha molecules to ethane and/or propane.

The molar ratio of hydrogen to hydrocarbon depends on the feed type including paraffin, olefin, naphthene or aromatics, the feed molecular carbon number, and the desired product between predominantly ethane, predominantly propane or ethane and propane of comparable abundance. For example, converting 1 mole of propane to ethane at stoichiometry, the process would require co-feeding 0.5 moles of hydrogen. In practice, the process can operate above or below this stoichiometry of 0.5 such as 0.33 to achieve greater than 40% ethane and less than 15% methane, depending on the process design parameters such as, feed contaminants, reactor type (fixed bed, moving bed, fluidized bed), and regeneration frequency. As the carbon number of feed molecules increases from light naphtha (C4-C7) to full range naphtha (C6-C10) the amount of hydrogen required for the reaction increases. For example, it would require 3.5 moles hydrogen and 2.0 moles of hydrogen to fully convert 1 mole of nonane to ethane and propane, respectively. The disclosed process can operate at three to five times the hydrogen-to-hydrocarbon ratio required to stoichiometrically convert the feed molecules to ethane and propane, respectively. It can also operate at 50% of hydrogen-to-hydrocarbon ratio required to stoichiometrically convert the feed molecules to ethane and propane, respectively. The hydrogen-to-hydrocarbon ratio would also depend on the need to produce petrochemical aromatics such as benzene, toluene and xylene.

The NEP catalyst for converting naphtha to ethane and propane may contain a molecular sieve comprising large or medium pore mouths, that is, comprising 10 or 12 member rings, respectively. Examples of suitable molecular sieves include MFI, MEL, MFI/MEL intergrowth, MTW, TUN, UZM-39, IMF, UZM-44, UZM-54, MWW, UZM-37, UZM-8, UZM-8HS. Examples of suitable molecular sieves further include FER, AHT, AEL (SAPO-11), AFO (SAPO-41), MRE, MFS, EUO-1, TON (ZSM-22), MTT (ZSM-23) and UZM-53. Additional molecular sieves with larger pores include FAU, EMT, FAU/EMT intergrowth, UZM-14, MOR, BEA, UZM-50, MTW, ZSM-12. Additional examples include MSE and UZM-35.

MFI is a suitable NEP catalyst. It will be appreciated that ZSM-5 is an MFI-type aluminosilicate zeolite belonging to the pentasil family of zeolites and having a chemical formula of NaₙAlₙSi₉₆-nO₁₉₂·16H₂O (0<n<10). In various embodiments, the ZSM-5 zeolite may comprise a silica-to-alumina molar ratio of 20 to 1000, 20 to 800, 20 to 600, 20 to 400, 20 to 200 or 20 to 80. In various embodiments, the ZSM-5 zeolite may comprise a crystal size in the range of 10 to 600 nm, 20 to 500 nm, 30 to 450, 40 to 400 nm, or 50 to 300 nm.

The NEP catalyst may comprise a bound zeolite. The binder may comprise an oxide of aluminum, silicon, zinc, titanium, zirconium and mixtures of thereof. The binder may comprise a phosphate in the binder or a phosphate of the forenamed oxide binder materials. Preferably, the binder is a silicon oxide. The MFI zeolite may be supported in a silicon oxide containing binder or an alumina containing binder such as aluminum phosphate.

MFI zeolite slurry may be first mixed with a binder in the form of colloidal suspension (sol) and gelling reagent and then dropped into hot oil to make spheres controlled to produce 1/888-inch to about 1/32-inch diameter calcined supports. Alternatively, the zeolite may be mixed with a silicon oxide containing binder and extruded to 1/32 to ¼ inch diameter extrudates. Extrudates may be washed with ammonia to remove sodium ions from the zeolite, dried and calcined to remove the organic structural directing agent (OSDA) from the synthesized zeolite. Optionally, the calcined support may be ammonium-ion exchanged using an ammonium nitrate solution to remove residual sodium ions and dried at about 110° C.

The NEP catalyst comprises a metal on the catalyst. The metal may comprise a transition metal. In a further example, the metal may comprise platinum, palladium, iridium, rhenium, ruthenium and mixtures thereof. The metal may be a noble metal. A modifier metal may also be included on the catalyst. The modifier metal may include tin, germanium, gallium, indium, thallium, zinc, silver and mixtures thereof. The modifier metal should be more concentrated on the binder than on the zeolite. About 0.01 to about 5 wt % of each of the transition metal and the modifier metal may be on the catalyst.

Metal may be incorporated into the binder by evaporative impregnation. A solution of platinum such as tetraamine platinate nitrate or chloroplatinic acid may be contacted with the bound spherical or extrudate supports which have been calcined and ion-exchanged in a rotary evaporator, followed by drying and oxidation.

The NEP catalyst comprises a metal on the bound spherical or extrudate supports of the catalyst. Preferably, more of the metal is on the binder than on the zeolite. At least 60 wt %, suitably at least 70 wt %, preferably at least 80 wt % and most preferably at least 90 wt % of the metal is on the binder. The zeolite and/or the entire NEP catalyst is steamed oxidized to drive the metal off the zeolite. Steaming is preferably effected after the metal is added to the catalyst. The dried, impregnated spherical or extrudate supports may be steam oxidized in air for sufficient time to provide NEP catalysts. Steam oxidation in air at a temperature of about 500° C. to about 650° C. and about 5 mol % to about 30 mol % steam for about 1 to 3 hours may be suitable.

The NEP catalysts must be reduced to activate them for catalyzing the NEP reaction. For example, the catalyst may be reduced in flowing hydrogen at about 500 to about 550° C. for 3 hours before contacting feed.

In accordance with the present disclosure, the first NEP reactor 130, the second NEP reactor 140, the third NEP reactor 150, and the fourth NEP reactor 160 may comprise similar or different NEP catalysts.

After conversion, a first contacted stream is discharged from the first NEP reactor 130 in line 131. The first contacted stream in line 131 may be passed to a heat exchanger 10 to cool the stream, perhaps generating steam. A cooled first contacted stream is taken in line 132 from the heat exchanger 10 and passed to the second NEP reactor 140. Alternatively, the heat removal between the stages may include producing superheated steam or heating of another process stream within or outside the current process. The second charge stream in line 109 is also passed to the second NEP reactor 140. The second charge stream in line 109 may be heated in the heat exchanger system 177 to provide a heated second charge stream in line 126 which is passed to the second NEP reactor 140. In an exemplary embodiment, the second charge stream in line 109 is passed to the second heat exchanger 50 to provide a second heated charge stream in line 126. The second heat exchanger 50 may comprise a hot inlet side 51 and a cold outlet side 53 for the hot stream, and a cold inlet side 52 and a hot outlet side 54 for the cold stream. A hot contacted stream is passed to the hot inlet side 51 and after heat exchange is removed from the cold outlet side 53 of the second heat exchanger 50. The second charge stream in line 109 is passed to the cold inlet side 52 and after heat exchange, the second heated charge stream is removed from the hot outlet side 54 of the second heat exchanger 50 in line 126.

In an exemplary embodiment, the heat exchanged second charge stream in line 126 may be combined with the heat exchanged first contacted stream in line 132 to provide a combined second charge stream in line 133 which is passed to the second NEP reactor 140. In the second NEP reactor 140, the first contacted stream and the second charge stream are contacted with a NEP catalyst to provide a second contacted stream in line 142. The NEP catalyst in the second NEP reactor 140 can be similar or a different NEP catalyst than the first NEP reactor 130.

After conversion, the second contacted stream is taken in line 142 from the second NEP reactor 140. The second contacted stream in line 142 may be passed to a heat exchanger 20 to generate steam. A heat exchanged perhaps cooled second contacted stream is taken in line 143 from the heat exchanger 20 and passed to the third NEP reactor 150. Alternatively, the heat removal between the stages may be via superheating of steam or heating of another process stream within or outside the current process. The third charge stream in line 116 is also passed to the third NEP reactor 140. The third charge stream in line 116 may be heated in the heat exchanger system 177 to provide a heat exchanged perhaps heated third charge stream in line 127 which is passed to the third NEP reactor 150. In an exemplary embodiment, the third charge stream in line 116 is passed to the third heat exchanger 60 to provide a third heated charge stream in line 127. The third heat exchanger 60 may comprise a hot inlet side 61 and a cold outlet side 63 for the hot stream, and a cold inlet side 62 and a hot outlet side 64 for the cold stream. A hot contacted stream is passed to the hot inlet side 61 and after heat exchange is removed from the cold outlet side 63 of the third heat exchanger 60. The third charge stream in line 116 is passed to the cold inlet side 62 and after heat exchange, the third heated charge stream is removed from the hot outlet side 64 of the third heat exchanger 60 in line 127.

In an exemplary embodiment, the third heated charge stream in line 127 may be combined with the heat exchanged second contacted stream in line 143 to provide a combined third charge stream in line 144 which is passed to the third NEP reactor 150. In the third NEP reactor 150, the second contacted stream and the third charge stream are contacted with an NEP catalyst to provide a third contacted stream in line 152. The NEP catalyst in the third NEP reactor 150 can be similar or a different NEP catalyst than the first NEP reactor 130 and the second NEP reactor 140.

After conversion, the third contacted stream is taken in line 152 from the third NEP reactor 140. The third contacted stream in line 152 may be passed to a heat exchanger 30 to generate steam. A heat exchanged perhaps cooled third contacted stream is taken in line 153 from the heat exchanger 30 and passed to the fourth NEP reactor 160. Alternatively, the heat removal between the stages may be via superheating of steam or heating of another process stream within or outside the current process. The fourth charge stream in line 118 is also passed to the fourth NEP reactor 160. The fourth charge stream in line 118 may be heated in the heat exchanger system 177 to provide a heat exchanged perhaps heated fourth charge stream in line 128 which is passed to the fourth NEP reactor 160. In an exemplary embodiment, the fourth charge stream in line 118 is passed to the fourth heat exchanger 70 to provide a fourth heated charge stream in line 128. The fourth heat exchanger 70 may comprise a hot inlet side 71 and a cold outlet side 73 for the hot stream, and a cold inlet side 72 and a hot outlet side 74 for the cold stream. A hot contacted stream is passed to the hot inlet side 71 and after heat exchange is removed from the cold outlet side 73 of the fourth heat exchanger 70. The fourth charge stream in line 118 is passed to the cold inlet side 72 and after heat exchange, the fourth heated charge stream is removed from the hot outlet side 74 of the fourth heat exchanger 70 in line 128.

In an exemplary embodiment, the heat exchanged fourth charge stream in line 128 may be combined with the heat exchanged third contacted stream in line 153 to provide a combined fourth charge stream in line 154 which is passed to the fourth NEP reactor 160. In the fourth NEP reactor 160, the third contacted stream and the fourth charge stream are contacted with an NEP catalyst to provide a fourth contacted stream in line 161. The NEP catalyst in the fourth NEP reactor 160 can be similar or a different NEP catalyst than the first NEP reactor 130, the second NEP reactor 140, and the third NEP reactor 160. In accordance with the present disclosure, the fourth contacted stream in line 161 may be a last stage or reactor contacted stream.

The present disclosure also includes preheating the dedicated naphtha streams before charging them to their dedicated NEP reactors. The first naphtha stream in line 103, the second naphtha stream in line 104, the third naphtha stream in line 105, and the fourth naphtha stream in line 106 are heat exchanged with one or more of the contacted NEP reactor effluent streams. In accordance with the present disclosure, the first naphtha stream in line 103, the second naphtha stream in line 104, the third naphtha stream in line 105, and the fourth naphtha stream in line 106 are heat exchanged with a contacted stream from the last NEP reactor of the multistage NEP reactor. In an exemplary embodiment, the first naphtha stream in line 103 is heated by heat exchange with the first hot contacted stream in line 162, the second naphtha stream in line 104 is heated by heat exchange with the second hot contacted stream in line 163, the third naphtha stream in line 105 is heated by heat exchange with the third hot contacted stream in line 164, and the fourth naphtha stream in line 106 is heated by heat exchange with the fourth hot contacted stream in line 165 in the heat exchanger system 177. The first hot contacted stream in line 162, the second hot contacted stream in line 163, the third hot contacted stream in line 164 and the fourth hot contacted stream in line 165 are all taken from the fourth contacted stream in line 161.

After paraffin conversion, a light paraffin stream is discharged from the fourth NEP reactor 160 in the fourth contacted stream in line 161. In accordance with an aspect of the present disclosure, the fourth contacted stream in line 161 is split into the first hot contacted stream in line 162, the second hot contacted stream in line 163, the third hot contacted stream in line 164 and the fourth hot contacted stream in line 165 for heating the respective charge streams in the heat exchanger system 177.

In an aspect, the fourth contacted stream in line 161 may be separated into a plurality of streams for heating the dedicated charge streams in the heat exchanger system 177. In an exemplary embodiment, the fourth contacted stream in line 161 may be separated into five hot streams, a first hot contacted stream in line 162, a second hot contacted stream in line 163, a third hot contacted stream in line 164, a fourth hot contacted stream in line 165, and a fifth hot contacted stream in line 166. The first hot contacted stream in line 162 is passed to the hot inlet side 41 of the first heat exchanger 40 to heat the first charge stream in line 108. A cooled first contacted stream is removed from the cold outlet side 43 of the first heat exchanger 40 in line 167. The second hot contacted stream in line 163 is passed to the hot inlet side 51 of the second heat exchanger 50 to heat the second charge stream in line 109. A cooled second contacted stream is removed from the cold outlet side 53 of the second heat exchanger 50 in line 168. The third hot contacted stream in line 164 is passed to the hot inlet side 61 of the third heat exchanger 60 to heat the third charge stream in line 116. A cooled third contacted stream is removed from the cold outlet side 63 of the third heat exchanger 60 in line 169. The fourth hot contacted stream in line 165 is passed to the hot inlet side 71 of the fourth heat exchanger 70 to heat the fourth charge stream in line 118. A cooled fourth contacted stream is removed from the cold outlet side 73 of the fourth heat exchanger 70 in line 171. The cooled first contacted stream in line 167, the cooled second contacted stream in line 168, the cooled third contacted stream in line 169, and the cooled contacted stream in line 171 are combined to provide a cooled light paraffin stream in line 173.

The light paraffin stream may comprise at least about 40 wt % ethane or at least about 40 wt % propane or at least about 70 wt % and preferably at least about 80 wt % ethane and propane. The ethane to propane ratio can range from about 0.1 to about 5. The light paraffin stream can have less than about 16 wt %, suitably less than about 14 wt %, preferably less than about 12 wt %, and more preferably less than about 10 wt % methane.

The cooled light paraffin stream in line 173 may be fed to the splitter column 170. In an exemplary embodiment, the splitter column 170 is a fractionation column that separates ethane and propane from aromatics. In the splitter column 170, the ethane and propane along with other light gases may be separated in an overhead stream in line 174. The aromatics are separated into a bottoms stream in line 175. The bottoms stream in line 175 may comprise single or multi-ring aromatics. In an aspect of the present disclosure, a portion of the bottoms aromatics stream can be recycled to the NEP reactor 151. In an exemplary embodiment, the bottoms stream in line 175 is separated into an aromatics product stream in line 176 and an aromatics recycle stream in line 178. The aromatics recycle stream in line 178 is recycled to the NEP reactor 151. The aromatics recycle stream in line 178 may be heat exchanged with the contacted reactor effluent stream in the heat exchanger system 177 to provide a preheated aromatics recycle stream in line 122. In an embodiment, the heat exchanger system 177 may comprise five dedicated heat exchangers. In an exemplary embodiment, the heat exchanger system 177 comprises a fifth dedicated heat exchanger 80 for heating the aromatics recycle stream in line 178. The fifth heat exchanger 80 may comprise a hot inlet side 81 and a cold outlet side 83 for the fifth hot contacted stream in line 166, and a cold inlet side 82 and a hot outlet side 84 for the aromatics recycle stream in line 178. The aromatics recycle stream in line 178 may be heated with a reactor effluent stream in the fifth heat exchanger 80. In an embodiment, the fifth hot contacted stream in line 166 is passed to the hot inlet side 81 of the fifth heat exchanger 80. A cooled fifth contacted stream is removed from the cold outlet side 83 of the fifth heat exchanger 80 in line 172. The cooled fifth contacted stream in line 172 is combined with the other four heat exchanged contacted streams to provide the cooled light paraffin stream in line 173.

A preheated aromatics recycle stream is removed from the hot outlet side 84 of the fifth heat exchanger 80 in line 122. The preheated aromatics recycle stream in line 122 may be further heated in a fired heater 123 to provide a heated aromatics recycle stream in line 124. The heated aromatics recycle stream in line 124 may be passed to the first NEP reactor in line 125 and to absorb the exotherm generated in the NEP reactors as previously described. The aromatics stream passes through all of the NEP reactors in series.

In an aspect of the present disclosure, the heat exchanger system may comprise a combined heat exchanger for heating the dedicated charge streams in a single heat exchanger.

Referring back to the splitter column 170, the overhead stream in line 174 is passed to an NEP separation unit 180 to separate ethane and propane. The NEP separation unit 180 may be a fractionation column or a series of fractionation columns and other separation units that may separate the overhead stream in line 174 into the hydrogen stream in line 181, an ethane stream in line 182, a propane stream in line 183 and the heavy stream in line 184. The NEP separation unit 180 may comprise a compressor to increase pressure suitable for downstream separation. The NEP separation unit 180 may comprise a demethanizer column that separates the overhead light paraffin stream into a gas stream in an overhead line and a C2+ paraffin stream in a bottoms line. The gas stream may be sent to a hydrogen purification unit such as a PSA unit to recover hydrogen in line 181 for recycle to the NEP reactor 151. Remaining methane from the hydrogen purification unit may be used for fuel gas. The C2+ paraffin stream may then be fed to a deethanizer column to produce the ethane stream in a deethanizer overhead line 182 and a C3+ paraffin stream in a deethanized bottoms line. The C3+ paraffin stream may then be fed to a depropanizer column to produce the propane stream in a depropanizer overhead line 183 and the heavy paraffin stream in the recycle line 184 which may comprise C4+ hydrocarbons. The NEP separation unit 180 may take other forms.

For example, the NEP separation unit 180 may omit a demethanizer column and the overhead light paraffin stream in line 174 may feed a deethanizer column which produces a C2− stream in a deethanizer overhead line. The C2− stream can be separated in the hydrogen purification unit to recover a hydrogen stream in line 181 while residual ethane and methane from the hydrogen purification unit can comprise or supplement the ethane stream in line 182. The hydrogen purification unit may comprise a membrane unit and the hydrogen recovered from the membrane unit may be further purified in an absorption column before it is recycled to the NEP reactor 151 in line 111. In an additional alternative, the C2− stream from the deethanizer column may be charged to an ethylene producing unit (not shown) in which ethane is converted to ethylene but methane and hydrogen rides through inertly to be recovered in a downstream ethylene recovery unit.

The ethane stream in line 182 may be charged to an ethylene producing unit in which ethane in the ethane stream is converted into ethylene. In an embodiment, the ethylene producing unit is a steam cracking unit. The ethane stream in line 182 may be cracked under steam in a furnace to produce a cracked stream including an ethylene stream. The ethane stream may be charged to the ethane steam cracking unit in the gas phase. The ethane steam cracking unit may preferably be operated at a temperature of about 750° C. (1382° F.) to about 950° C. (1742° F.). The cracked stream exiting the furnace of the ethane steam cracking unit may be in a superheated state. One or more quench columns, or other devices known in the art, but preferably an oil quench column and/or a water quench column, may be used for quenching or separating the cracked stream into a plurality of cracked streams. The ethane steam cracking unit may further comprise additional distillation columns, amine wash columns, compressors, expanders, etc. to separate the cracked stream into cracked streams rich in individual light olefins the most predominant of which is the ethylene stream. The ethylene stream may comprise a yield of at least 75 wt %, preferably at least 80 wt %, ethylene based on the ethane stream in line 182. Among the other components in the cracked stream exiting the ethane steam cracking, ethylene producing unit may be hydrogen, methane, propylene, butene, and pyrolysis gas. Each of these components may be recovered and further processed.

The ethylene stream and a propylene stream from the ethylene producing unit may be recovered or transported to polymerization plants, chemical plants or exported. A butene stream may be recovered and used to produce plastics or other petrochemicals by processes such as polymerization or exported. Product recovery of at least 50 wt %, typically at least 60 wt % and suitably at least 70 wt % of valuable ethylene, propylene, and butylene products is achievable from the ethane steam cracking unit 30 based on the ethane stream in line 32.

The propane stream in line 183 may be charged to a propylene producing unit (not shown) in which propane in the propane stream is converted into propylene. The propylene producing unit may be a propane dehydrogenation (PDH) unit. PDH catalyst is used in a dehydrogenation reaction process to catalyze the dehydrogenation of propane, such as propane. The conditions in the dehydrogenation reactor may include a temperature of about 500 to about 800° C., a pressure of about 40 to about 310 kPa (abs) and a catalyst to oil ratio of about 5 to about 100.

The dehydrogenation reaction may be conducted in a fluidized manner such that gas, which may comprise the reactant paraffins with or without a fluidizing inert gas, is distributed to the reactor in a way that lifts the dehydrogenation catalyst in the reactor vessel while catalyzing the dehydrogenation of paraffins. During the catalytic dehydrogenation reaction, coke is deposited on the dehydrogenation catalyst leading to reduction of the activity of the catalyst. The dehydrogenation catalyst must then be regenerated in a regenerator. The regenerator may combust coke from the dehydrogenation catalyst and fuel gas to ensure sufficient enthalpy in the dehydrogenation reactor to promote the endothermic reaction.

The dehydrogenation catalyst selected should minimize cracking reactions and favor dehydrogenation reactions. Suitable catalysts for use herein include an active metal which may be dispersed in a porous inorganic carrier material such as silica, alumina, silica alumina, zirconia, or clay. An exemplary embodiment of a catalyst includes alumina or silica-alumina containing gallium, a noble metal, and an alkali or alkaline earth metal.

The catalyst support comprises a carrier material, a binder and an optional filler material to provide physical strength and integrity. The carrier material may include alumina or silica-alumina. Silica sol or alumina sol may be used as the binder. The alumina or silica-alumina generally contains alumina of gamma, theta and/or delta phases. The catalyst support particles may have a nominal diameter of about 400 to about 5000 micrometers with the average diameter of about 600 to about 3500 micrometers. Preferably, the surface area of the catalyst support is about 85 to about 140 m2/g.

The fluidized dehydrogenation catalyst may comprise a dehydrogenation metal on a support. The dehydrogenation metal may be a one or a combination of transition metals. A noble metal may be a preferred dehydrogenation metal such as platinum or palladium. Gallium is an effective metal for paraffin dehydrogenation. Metals may be deposited on the catalyst support by impregnation or other suitable methods or included in the carrier material or binder during catalyst preparation.

The acid function of the catalyst should be minimized to prevent cracking and favor dehydrogenation. Alkali metals and alkaline earth metals may also be included in the catalyst to attenuate the acidity of the catalyst. Rare earth metals may be included in the catalyst to control the activity of the catalyst. Concentrations of 0.001% to 10 wt % metals may be incorporated into the dehydrogenation catalyst. In the case of the noble metals, it is preferred to use about 10 parts per million (ppm) by weight to about 600 ppm by weight noble metal. More preferably it is preferred to use about 10 to about 100 ppm by weight noble metal. The preferred noble metal is platinum. Gallium should be present in the range of 0.3 wt % to about 3 wt %, preferably about 0.5 wt % to about 2 wt %. Alkali and alkaline earth metals may be present in the range of about 0.05 wt % to about 1 wt %.

Regenerated catalyst may be contacted with the propane stream perhaps with a fluidizing gas to lift the propane stream and dehydrogenation catalyst up a riser while dehydrogenation occurs. Above the riser spent dehydrogenation catalyst and propylene product may be separated by a centripetal separation device. Propylene product gas may be quenched with a cooling fluid to prevent over reaction to undesired by-products. Separation of the propylene product may include quench contacting and fractionation to produce a propylene product stream. Unreacted propane may be recycled to the dehydrogenation reactor and lighter gases may be recycled to the regenerator as fuel gas to be combusted to provide enthalpy for the reaction.

The propylene producing unit may also employ a catalytic moving bed reactor. The reactor section may comprise several radial flow reactors in parallel or series heated by charge and interstage heaters. The propane stream perhaps with added hydrogen flows in each dehydrogenation reactor from a screened center pipe through an annular dehydrogenation catalyst bed to an outer effluent annulus. Flow may be in the reverse fashion. The dehydrogenation catalyst may comprise a noble metal or mixtures thereof, a modifier selected from the group consisting of alkali metals or alkaline-earth metals and mixtures thereof, a component selected from the group consisting of tin, germanium, lead, indium, gallium, thallium, and mixtures thereof, and a porous support forming a catalyst particle. The catalyst support may comprise oil dropped alumina spheres.

Dehydrogenation conditions may include a temperature of from about 400 to about 900° C., a pressure of from about 0.01 to 10 atmospheres absolute, and a liquid hourly space velocity (LHSV) of from about 0.1 to 100 hr−1. The pressure in the dehydrogenation reactor is maintained as low as practicable, consistent with equipment limitations, to maximize chemical equilibrium advantages. Spent dehydrogenation catalyst in the annular catalyst bed may be withdrawn from the bottom of the bed, forwarded to a regenerator to combust coke from the catalyst with air at about 450 to about 600° C. Noble metal on the catalyst may be redispersed by an oxyhalogenation process, dried and returned to the top of the dehydrogenation catalyst bed as regenerated dehydrogenation catalyst.

Dehydrogenation effluent from the propylene producing unit 40 may be cooled, compressed, dried and hydrogen is cryogenically separated from the hydrocarbons with a net gas purity of 85 to 93 mol % hydrogen. Hydrocarbon liquid is selectively hydrogenated to convert diolefins and acetylenes and the hydrocarbon liquid is fractionated in a deethanizer column to remove ethane and propylene is split from propane in a propane-propylene splitter column to provide polymer-grade propylene. Propane may be recycled as feed to the propylene producing unit.

The heavy stream in line 184 which may be taken from a bottom of a depropanizer column may comprise C4+ paraffins. The heavy stream in line 184 may be recycled to the NEP reactor 151 to produce more ethane and propane.

The foregoing disclosure provides a process for converting naphtha to ethane and propane feed with maximizing the ethane to propane ratio.

### EXAMPLES

### Example 1

A naphtha feed was used as a feed for NEP reactor. The composition of the naphtha feed is as below in Table 1:

TABLE 1

| Constituents | wt % |
|---|---|
| Propane | 0.20 |
| n-Butane | 10.80 |
| i-Butane | 28.83 |
| n-Pentane | 5.88 |
| i-Pentane | 21.37 |
| n-Hexane | 4.88 |
| 2-methyl pentane | 21.78 |
| n-Heptane | 2.08 |
| 3-methyl hexane | 1.54 |
| Cyclopentane | 0.14 |
| Cyclohexane | 0.46 |
| Methyl cyclopentane | 1.37 |
| Ethyl cyclopentane | 0.56 |
| Benzene | 0.11 |
| Total | 100.00 |

A comparative study was performed to demonstrate and compare the current process with other processes in terms of yield. A total of five tests A, B, C, D, and E were performed. In test A, the naphtha feed was passed to a single stage NEP reactor without recycling aromatics diluent stream. For Test B, the naphtha feed was passed to a single stage NEP reactor with the recycled aromatics stream. Tests C and D were conducted in a multistage reactor with four stages where all the naphtha is fed to stage 1. For tests C and D only hydrogen was staged to the four reactors meaning hydrogen was separated and passed to each dedicated reactor. Test E represents the current process. In test E, both naphtha feed and hydrogen were staged in all the four reactors. The various parameters of tests A to E are shown in Table 2 below:

TABLE 2

| | | TEST | | | | |
|---|---|---|---|---|---|---|
| | | A | B | C | D | E |
| | | Description | | | | |
| | | | | No | No Aromatics | Aromatics |
| | | | | Aromatics | Recycle | Recycle and |
| | | No | | Recycle | All Naphtha | both |
| | | Aromatics | Aromatics | All Naphtha | to Reactor 1 | Naphtha and |
| | Unit | Recycle | Recycle | to Reactor 1 | and H2 staged | H2 staged |
| No. of stages | | 1 | 1 | 4 | 4 | 4 |
| Aromatics/Feed | w/w | 0 | 0.4 | 0.0 | 0.0 | 0.4 |
| Diluent mole | kg/kg | NA | 88.6 | NA | NA | 96.4 |
| weight | mol | | | | | |
| Naphtha mole | kg/kg | 69.4 | 69.4 | 69.4 | 69.4 | 69.4 |
| weight | mol | | | | | |
| Overall | mol/ | 1.20 | 1.20 | 1.2 | 1.2 | 1.2 |
| H2/Naphtha | mol | | | | | |
| Naphtha split | % | 100.0 | 100.0 | 100.0 | 100.0 | 19.0 |
| to Stage 1 | | | | | | |
| Naphtha split | % | NA | NA | 0.0 | 0.0 | 20.0 |
| to Stage 2 | | | | | | |
| Naphtha split | % | NA | NA | 0.0 | 0.0 | 25.0 |
| to Stage 3 | | | | | | |
| Naphtha split | % | NA | NA | 0.0 | 0.0 | 36.0 |
| to Stage 4 | | | | | | |
| Hydrogen split | % | 100.0 | 100.0 | 25.0 | 25.2 | 18.8 |
| to Stage 1 | | | | | | |
| Hydrogen split | % | NA | NA | 25.0 | 24.4 | 19.3 |
| to Stage 2 | | | | | | |
| Hydrogen split | % | NA | NA | 25.0 | 28.6 | 25.9 |
| to Stage 3 | | | | | | |
| Hydrogen split | % | NA | NA | 25.0 | 21.8 | 36.1 |
| to Stage 4 | | | | | | |
| Overall Naphtha | kg/h | 2.0 | 2.0 | 2.0 | 2.0 | 2.0 |
| WHSV | Nap/kg | | | | | |
| | cat | | | | | |
| Catalyst | | | | | | |
| Distribution | | | | | | |
| Stage 1 | % | 100 | 100 | 56.9 | 56.0 | 10.4 |
| Stage 2 | % | NA | NA | 15.5 | 15.9 | 16.6 |
| Stage 3 | % | NA | NA | 14.5 | 14.9 | 28.0 |
| Stage 4 | % | NA | NA | 13.2 | 13.2 | 45.0 |

All the tests were conducted at the same reactor inlet conditions except test D. In test D, the feed temperatures were increased to yield a similar naphtha conversion as in test E for direct comparison. The temperatures of the reactors and the results for tests A to E are shown in Table 3 below:

TABLE 3

| | | TEST | | | | |
|---|---|---|---|---|---|---|
| | | | B | C | D | |
| | | A | Single | 4 Stages | 4 Stages | E |
| | | Single | Stage | No Recycle | No Recycle | 4 Stages |
| | | Stage No | Including | All Naphtha | All Naphtha | Including |
| | Unit | Recycle | Recycle | to Reactor 1 | to Reactor 1 | Recycle |
| Stage 1 Inlet | ° C. | 400.0 | 400.0 | 400.0 | 421.5 | 400.0 |
| Temperature | | | | | | |
| Stage 1 Outlet | ° C. | 660.2 | 582.4 | 409.2 | 427.5 | 465.3 |
| Temperature | | | | | | |
| Stage 1 | ° C. | 260.2 | 182.4 | 9.2 | 6.0 | 65.3 |
| Temperature Rise | | | | | | |
| Stage 2 Inlet | ° C. | | | 438.9 | 460.4 | 438.9 |
| Temperature | | | | | | |
| Stage 2 Outlet | ° C. | | | 459.8 | 495.5 | 501.7 |
| Temperature | | | | | | |
| Stage 2 | ° C. | | | 20.9 | 35.1 | 62.8 |
| Temperature Rise | | | | | | |
| Stage 3 Inlet | ° C. | | | 450.9 | 472.4 | 450.9 |
| Temperature | | | | | | |
| Stage 3 Outlet | ° C. | | | 460.6 | 481.7 | 508.5 |
| Temperature | | | | | | |
| Stage 3 | ° C. | | | 9.8 | 9.4 | 57.6 |
| Temperature Rise | | | | | | |
| Stage 4 Inlet | ° C. | | | 450.9 | 472.4 | 450.9 |
| Temperature | | | | | | |
| Stage 4 Outlet | ° C. | | | 460.6 | 481.7 | 508.5 |
| Temperature | | | | | | |
| Stage 4 | ° C. | | | 9.8 | 9.4 | 57.6 |
| Temperature Rise | | | | | | |
| Naphtha | wt % | 99.9 | 99.3 | 93.3 | 96.7 | 96.7 |
| Conversion | | | | | | |
| Hydrogen | % | 97.6 | 97.2 | 34.7 | 39.7 | 84.5 |
| Conversion | | | | | | |
| Selectivity | | | | | | |
| Methane | wt % | 58.4 | 22.1 | 5.3 | 7.7 | 12.5 |
| Ethane | wt % | 11.9 | 59.9 | 32.4 | 46.5 | 58.8 |
| Propane | wt % | 0.4 | 3.7 | 46.5 | 26.4 | 17.6 |
| Aromatics | wt % | 29.3 | 14.3 | 15.8 | 19.3 | 11.0 |
| Total | | 100.0 | 100.0 | 100.0 | 99.9 | 99.9 |
| Ethane + Propane | wt % | 12.30 | 63.60 | 78.9 | 72.9 | 76.4 |
| (E + P) | | | | | | |
| Ethane/Propane | wt/wt | 29.75 | 16.19 | 0.7 | 1.8 | 3.3 |
| (E/P) | | | | | | |

As shown in Table 3, test A with single stage operation resulted in an excessive exotherm resulting in excessively high methane and aromatics selectivity at the sacrifice of having lower ethane plus propane selectivity. Test B with single stage and aromatics recycle showed the advantage of using recycled aromatics to decrease the exotherm. Test B showed improved ethane plus propane selectivity as compared to test A by reducing the selectivity to methane and aromatics. For test C with multistage reactor, all naphtha to first stage and staging hydrogen operation, results showed that staging hydrogen addition can be effective at increasing combined ethane and propane over that for single stage operation. However, the downside for test C was that naphtha conversion was lower and hydrogen conversion was extremely low as shown in Table 3. Also, for test C the ethane to propane ratio was also extremely low. Methane selectivity was low for test C. Test D showed that at higher naphtha conversion, combined ethane plus propane selectivity greatly decreased and hydrogen conversion remained low. Further, test D results showed that the aromatic selectivity was greater than that for the single stage operation with recycle. The ethane to propane ratio for test D was increased but is still low compared to other cases.

Test E illustrates the advantages of the current process configuration utilizing staged hydrogen and staged naphtha addition with recycled aromatics. It is evident from the results in Table 4 that the aromatics selectivity for test E was lowest as compared to other tests A to D. Also, for test E the ethane to propane ratio was higher than that for the alternative staging tests C and D. Therefore, the process of the present disclosure provides a higher ethane to propane ratio as demonstrated by the test results shown in Table 3.

### Specific Embodiments

While the following is described in conjunction with specific embodiments, it will be understood that this description is intended to illustrate and not limit the scope of the preceding description and the appended claims.

A first embodiment of the invention is a process for converting naphtha to ethane and propane comprising separating a naphtha stream into a plurality of naphtha streams; contacting each of the plurality of naphtha streams and a hydrogen stream with a catalyst in a dedicated one of a plurality of reactors to produce a plurality of contacted streams; and separating one of the contacted streams into an ethane stream and a propane stream. An embodiment of the present disclosure is one, any or all of prior embodiments in this paragraph up through the first embodiment in this paragraph further comprising mixing a first contacted stream from a first reactor of the plurality of reactors with a second naphtha stream of the plurality of naphtha streams and contacting the second naphtha stream and the first contacted stream and a hydrogen stream with the catalyst in a second reactor of the plurality of reactors. An embodiment of the present disclosure is one, any or all of prior embodiments in this paragraph up through the first embodiment in this paragraph further comprising cooling the first contacted stream before contacting the first contacted stream in the second reactor. An embodiment of the present disclosure is one, any or all of prior embodiments in this paragraph up through the first embodiment in this paragraph further comprising separating the hydrogen stream into a plurality of hydrogen streams; and charging each of the plurality of hydrogen streams to a dedicated one of the plurality of reactors. An embodiment of the present disclosure is one, any or all of prior embodiments in this paragraph up through the first embodiment in this paragraph further comprising heat exchanging one naphtha stream of the plurality of naphtha streams with a contacted stream to preheat one naphtha stream and cool a contacted stream. An embodiment of the present disclosure is one, any or all of prior embodiments in this paragraph up through the first embodiment in this paragraph further comprising splitting a last contacted stream from the last reactor into a plurality of contacted streams and heat exchanging one of the contacted streams with a one of the plurality of naphtha streams. An embodiment of the present disclosure is one, any or all of prior embodiments in this paragraph up through the first embodiment in this paragraph further comprising heat exchanging all of the plurality of contacted streams with a dedicated one of the plurality of naphtha streams. An embodiment of the present disclosure is one, any or all of prior embodiments in this paragraph up through the first embodiment in this paragraph further comprising separating the plurality of contacted streams into a light paraffin stream and an aromatic stream. An embodiment of the present disclosure is one, any or all of prior embodiments in this paragraph up through the first embodiment in this paragraph wherein the plurality of reactors comprise four reactors. An embodiment of the present disclosure is one, any or all of prior embodiments in this paragraph up through the first embodiment in this paragraph further comprising mixing one of the plurality of hydrogen streams with one of the plurality of naphtha streams to provide a plurality of mixed streams; and charging each of the plurality of mixed stream to a dedicated one of the plurality of reactors. An embodiment of the present disclosure is one, any or all of prior embodiments in this paragraph up through the first embodiment in this paragraph further comprising mixing a second contacted stream from the second reactor of the plurality of reactors with a third naphtha stream of the plurality of naphtha streams and contacting the third naphtha stream and the second contacted stream and a hydrogen stream with the catalyst in a third reactor of the plurality of reactors.

A second embodiment of the present disclosure is a process for converting naphtha to ethane and propane comprising separating a naphtha stream into a plurality of naphtha streams; contacting each of the plurality of naphtha streams and a hydrogen stream with a catalyst in a dedicated one of a plurality of reactors to produce a plurality of contacted streams, wherein a first contacted stream from a first reactor of the plurality of reactors is mixed with a second naphtha stream of the plurality of naphtha streams and the third naphtha stream and the first contacted stream and a hydrogen stream are contacted with the catalyst in a second reactor of the plurality of reactors; and separating one of the plurality of contacted streams into an ethane stream and a propane stream. An embodiment of the present disclosure is one, any or all of prior embodiments in this paragraph up through the second embodiment in this paragraph further comprising cooling the first contacted stream before mixing with the second naphtha stream. An embodiment of the present disclosure is one, any or all of prior embodiments in this paragraph up through the second embodiment in this paragraph further comprising separating the hydrogen stream into a plurality of hydrogen streams; and charging each of the plurality of hydrogen streams to a dedicated one of the plurality of reactors. An embodiment of the present disclosure is one, any or all of prior embodiments in this paragraph up through the second embodiment in this paragraph further comprising heat exchanging one naphtha stream of the plurality of naphtha streams with a contacted stream to preheat one naphtha stream and cool a contacted stream.

A third embodiment of the present disclosure is a process for converting naphtha to ethane and propane comprising separating a naphtha stream into a plurality of naphtha streams; contacting each of the plurality of naphtha streams and a hydrogen stream with a catalyst in a dedicated one of a plurality of reactors to produce a plurality of contacted streams, wherein an effluent stream from a first reactor is fed to a second reactor of the plurality of reactors; separating one of the contacted streams into an ethane stream and a propane stream. An embodiment of the present disclosure is one, any or all of prior embodiments in this paragraph up through the third embodiment in this paragraph wherein the effluent stream is cooled before feeding to the second reactor. An embodiment of the present disclosure is one, any or all of prior embodiments in this paragraph up through the third embodiment in this paragraph further comprising separating the hydrogen stream into a plurality of hydrogen streams; and charging each of the plurality of hydrogen streams to a dedicated one of the plurality of reactors. An embodiment of the present disclosure is one, any or all of prior embodiments in this paragraph up through the third embodiment in this paragraph further comprising splitting a last contacted stream from a last reactor of the plurality of reactors into a plurality of contacted streams and heat exchanging one of the contacted streams with a one of the plurality of naphtha streams. An embodiment of the present disclosure is one, any or all of prior embodiments in this paragraph up through the third embodiment in this paragraph further comprising heat exchanging all of the plurality of contacted streams with a dedicated one of the plurality of naphtha streams.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for converting naphtha to ethane and propane comprising:
separating a naphtha stream into a plurality of naphtha streams;
contacting each of said plurality of naphtha streams and a hydrogen stream with a catalyst in a dedicated one of a plurality of reactors to produce a plurality of contacted streams; and
separating one of said contacted streams into an ethane stream and a propane stream.

2. The process of claim 1 further comprising mixing a first contacted stream from a first reactor of said plurality of reactors with a second naphtha stream of said plurality of naphtha streams and contacting said second naphtha stream and said first contacted stream and a hydrogen stream with said catalyst in a second reactor of said plurality of reactors.

3. The process of claim 2 further comprising cooling said first contacted stream before contacting said first contacted stream in said second reactor.

4. The process of claim 1 further comprising separating said hydrogen stream into a plurality of hydrogen streams; and charging each of said plurality of hydrogen streams to a dedicated one of said plurality of reactors.

5. The process of claim 1 further comprising heat exchanging one naphtha stream of the plurality of naphtha streams with a contacted stream to preheat one naphtha stream and cool a contacted stream.

6. The process of claim 5 further comprising splitting a last contacted stream from said last reactor into a plurality of contacted streams and heat exchanging one of said contacted streams with a one of said plurality of naphtha streams.

7. The process of claim 6 further comprising heat exchanging all of said plurality of contacted streams with a dedicated one of said plurality of naphtha streams.

8. The process of claim 7 further comprising:
separating the plurality of contacted streams into a light paraffin stream and an aromatic stream.

9. The process of claim 1 wherein said plurality of reactors comprise four reactors.

10. The process of claim 4 further comprising:
mixing one of said plurality of hydrogen streams with one of the plurality of naphtha streams to provide a plurality of mixed streams; and
charging each of said plurality of mixed stream to a dedicated one of said plurality of reactors.

11. The process of claim 2 further comprising mixing a second contacted stream from said second reactor of said plurality of reactors with a third naphtha stream of said plurality of naphtha streams and contacting said third naphtha stream and said second contacted stream and a hydrogen stream with said catalyst in a third reactor of said plurality of reactors.

12. A process for converting naphtha to ethane and propane comprising:
separating a naphtha stream into a plurality of naphtha streams;
contacting each of said plurality of naphtha streams and a hydrogen stream with a catalyst in a dedicated one of a plurality of reactors to produce a plurality of contacted streams, wherein a first contacted stream from a first reactor of said plurality of reactors is mixed with a second naphtha stream of said plurality of naphtha streams and said third naphtha stream and said first contacted stream and a hydrogen stream are contacted with said catalyst in a second reactor of said plurality of reactors; and
separating one of said plurality of contacted streams into an ethane stream and a propane stream.

13. The process of claim 12 further comprising cooling said first contacted stream before mixing with said second naphtha stream.

14. The process of claim 12 further comprising separating said hydrogen stream into a plurality of hydrogen streams; and charging each of said plurality of hydrogen streams to a dedicated one of said plurality of reactors.

15. The process of claim 12 further comprising heat exchanging one naphtha stream of said plurality of naphtha streams with a contacted stream to preheat one naphtha stream and cool a contacted stream.

16. A process for converting naphtha to ethane and propane comprising:
separating a naphtha stream into a plurality of naphtha streams;
contacting each of said plurality of naphtha streams and a hydrogen stream with a catalyst in a dedicated one of a plurality of reactors to produce a plurality of contacted streams, wherein an effluent stream from a first reactor is fed to a second reactor of said plurality of reactors;
separating one of said contacted streams into an ethane stream and a propane stream.

17. The process of claim 16 wherein said effluent stream is cooled before feeding to said second reactor.

18. The process of claim 16 further comprising:
separating said hydrogen stream into a plurality of hydrogen streams; and
charging each of said plurality of hydrogen streams to a dedicated one of said plurality of reactors.

19. The process of claim 16 further comprising splitting a last contacted stream from a last reactor of said plurality of reactors into a plurality of contacted streams and heat exchanging one of said contacted streams with a one of said plurality of naphtha streams.

20. The process of claim 19 further comprising heat exchanging all of said plurality of contacted streams with a dedicated one of said plurality of naphtha streams.
